(19) United States
(12) Patent Application Publication
(10) Pub. No.: US 2020/0290939 A1
(43) Pub. Date: Sep. 17, 2020

(54) METHODS AND SYSTEMS FOR OLEFIN PRODUCTION

(71) Applicant: SABIC Global Technologies B.V., Bergen op Zoom (NL)

(72) Inventor: Joris VAN WILLIGENBURG, Maastricht (NL)

(21) Appl. No.: 16/650,069

(22) PCT Filed: Nov. 12, 2018
(86) PCT No.: PCT/IB2018/058876

§ 371 Date: Mar. 24, 2020

Related U.S. Application Data
(60) Provisional application No. 62/585,181, filed on Nov. 13, 2017.

(51) Int. Cl. C07C 4/04; B01J 19/00; B01J 12/00

(52) U.S. Cl. CPC C07C 4/04; B01J 2219/00054; B01J 12/00; B01J 19/0013

(57) ABSTRACT

Methods and systems for improving energy conversion from the heat available in a hydrocarbon feedstream during the production of olefins. In a particular non-limiting embodiment, the method can include increasing the temperature of a hydrocarbon feedstream and a hydrogen gas feedstream via a first heat exchanger; combining the feedstreams and expanding the combined feedstream in an expander to decrease the pressure and/or temperature of the combined feedstream; increasing the temperature of the combined feedstream via a second heat exchanger; feeding the combined feedstream into a reactor to produce a reactor effluent; decreasing the temperature of the reactor effluent; and compressing the reactor effluent in a compressor, where the expansion of the combined feedstream drives the compressor.

### CROSS REFERENCE TO RELATED APPLICATIONS

This application claims the benefit of priority of U.S. Provisional Patent Application No. 62/585,181, filed Nov. 13, 2017, which is hereby incorporated by reference in its entirety.

### FIELD

The presently disclosed subject matter relates to methods and systems for the production of olefins and relates to techniques for improving energy conversion during the production of the olefins.

### BACKGROUND

Many petrochemical processes are endothermic processes and large amounts of energy can be required to drive such processes. In particular, the input of heat to perform such petrochemical processes can be greater than the amount of heat that is available through heat recovery, which can result in the consumption of high quality energy and be cost-intensive. For example, cracking of hydrocarbons to produce olefins is an endothermic process and requires energy, which can be in the form of high temperature heat to provide the required heat to drive the reaction within the reactor, and mechanical work, which can be used to drive the compressors used for product separation.

Methods and systems for reducing the amount of energy consumed during petrochemical processes are known in the art. For example, G.B. Patent No. 1,516,362 discloses a process for producing hydrogen-rich gas in a reformer furnace that includes the generation of electrical power and compressed air by passing furnace flue gas and hot combustion gases through the turbine side of an air compressor in common drive with a generator. WO 2007/143776 discloses a process for generating synthesis gas that includes using the outlet flue gas from a reactor to drive an expansion turbine. Indian Application No. 3225/CHE/2008 discloses a method of recovering energy from a fluid catalytic cracking (FCC) unit having a reactor and a regenerator, where a turbo-expander train can be used to combust and expand the generated syngas to drive a first compressor. The turbo-expander train can include a first expander coupled to a first compressor via a shaft, and the energy required to rotate the shaft can be extracted from the heated gas produced in the combustion zone by the first expander. Australian Patent No. 2001/292544 discloses a method to improve the efficiency of a reformer/fuel cell system by recapturing heat energy generated by the fuel cell to produce mechanical energy that can be used to drive an expander (e.g., turbine) downstream of the burner. U.S. Pat. No. 5,114,682 discloses a process for the recovery of heat energy during a FCC catalyst regeneration process that includes expanding the effluent gases from the catalyst regeneration process to obtain work energy that can be used to operate an expansion turbine-compressor. U.S. Pat. No. 3,401,124 discloses a method that includes recovering kinetic energy from a flue gas exiting a catalyst regeneration zone by expanding the flue gas to generate useful work that can drive an induction generator. EP0982539 discloses a method for the recovery of heat from flue gases to produce mechanical work that includes passing a flue gas through a turbine of a turbo charger/compressor to convert the thermal energy of the flue gas into mechanical energy. U.S. Pat. No. 4,154,055 discloses an energy recovery system useful for extracting energy from the combusted exhaust gases of a furnace.

There remains a need in the art for methods and systems that can reduce the amount of net energy that is consumed during the formation of olefin containing hydrocarbon streams.

### SUMMARY OF THE DISCLOSED SUBJECT MATTER

The presently disclosed subject matter provides methods and systems for olefin production from a hydrocarbon feedstream, e.g., by hydropyrolysis, and relates to techniques for improving energy conversion from the heat available in the hydrocarbon feedstream.

An example method for producing olefins using a hydrocarbon feedstream and a gas feedstream including hydrogen can include increasing the temperature of the hydrocarbon feedstream and the hydrogen gas feedstream by a first heat exchanger. The method can further include combining the feedstreams and expanding the combined feedstream in a reactants expander to decrease the pressure and/or temperature of the combined feedstream. In certain embodiments, the method can include increasing the temperature of the combined feedstream via a second heat exchanger and feeding the combined feedstream into a reactor to produce a reactor effluent. The method can include decreasing the temperature of the reactor effluent and compressing the reactor effluent in a compressor. The reactants expander and the compressor can be coupled and the expansion of the combined feedstream can drive the compressor. In certain embodiments, the method can further include expanding the reactor effluent in a reactor effluent expander prior to the reduction in the temperature of the reactor effluent. In certain embodiments where the reactor effluent is expanded, the reactor effluent expander can be coupled to the reactants expander and the compressor and the expansion of the reactor effluent can be used to drive the compressor. In other certain embodiments, the hydrocarbon feedstream can include ethane and/or the reactor effluent can include ethylene. In a non-limiting embodiment, the hydrocarbon feedstream can include propane and/or the reactor effluent can include propylene. In certain embodiments, the gas feedstream can further include a gas such as steam, nitrogen or a combination thereof. In certain embodiments, the hydrocarbon feedstream can include butane and/or the reactor effluent can include 1-butene, isobutylene, hydrogen or a combination thereof. In a non-limiting embodiment, the hydrocarbon feedstream can be a C4 hydrocarbon stream and/or the reactor effluent can include 1,3-butadiene. In certain embodiments, the gas feedstream and/or the hydrocarbon feedstream can have a pressure of about 10 to 50 bar absolute and/or a temperature of about 10° C. to about 100° C. prior to increasing the temperature of the hydrocarbon feedstream and the hydrogen gas feedstream via a first heat exchanger.

The presently disclosed subject matter further provides methods for producing ethylene using a feedstream including ethane and hydrogen. In certain embodiments, a method includes increasing the temperature of the feedstream via a first heat exchanger to a temperature equal to or greater than about 500° C. The method can further include expanding the feedstream in an expander to a pressure of about 4 bar absolute. In certain embodiments, the method can include increasing the temperature of the feedstream to about 500° C. to about 700° C. via a second heat exchange. The method can further include feeding the feedstream into a reactor and cracking the feedstream at a temperature from about 700° C. to about 880° C. to produce a reactor effluent comprising ethylene at a temperature from about 700° C. to about 880° C. and/or at a pressure from about 1 to about 5 bar absolute. In certain embodiments, the method can further include decreasing the temperature of the reactor effluent and compressing the reactor effluent in a compressor to a pressure from about 0.3 bar to about 35 bar. The method can include separating ethylene from the compressed reactor effluent to produce an ethylene product stream. In certain embodiments, the expander and the compressor are coupled and the expansion of the feedstream drives the compressor. In certain embodiments, the temperature of the reactor effluent is about 20° C. to 30° C. prior to the compression of the reactor effluent. In certain embodiments, a method includes increasing the temperature of the feedstream via a first heat exchanger and expanding the feedstream in a reactor feed expander. The method can include increasing the temperature of the feedstream in a second heat exchanger and feeding the feedstream into a reactor. The method can further include cracking the feedstream at a temperature from about 700° C. to about 880° C. in the reactor to produce a reactor effluent that includes ethylene. In certain embodiments, the method can further include decreasing the temperature of the reactor effluent and compressing the reactor effluent in a compressor, where the expander and the compressor are coupled and the expansion of the feedstream drives the compressor.

The presently disclosed subject matter further provides a system for producing olefins using a hydrocarbon feedstream and a gas feedstream including hydrogen. The system can include a first heat exchanger for increasing the temperature of the hydrocarbon feedstream and the hydrogen gas feedstream. The system can further include a reactants expander, coupled to the first heat exchanger, for decreasing the pressure and/or temperature of the feedstreams and for converting heat energy extracted from the feedstreams into mechanical work. In certain embodiments, the system can include a second heat exchanger, coupled to the expander, for increasing the temperature of the feedstreams via a second heat exchanger. The system can further include a reactor, coupled to the second heat exchanger, for producing a reactor effluent from the feedstreams, and a reactor effluent expander, coupled to the reactor and the first heat exchanger, for decreasing the temperature and/or pressure of the reactor effluent. The system can include a third heat exchanger, coupled to the first heat exchanger, for decreasing the temperature of the reactor effluent and a compressor, coupled to the third heat exchanger, for compressing the reactor effluent in a compressor. The reactants expander and the compressor within the system can be coupled and the expansion of the feedstreams can drive the compressor.

In the context of the present invention, twelve embodiments are now described. Embodiment 1 is method for producing olefins using a hydrocarbon feedstream and a gas feedstream including hydrogen. The method includes (a) increasing the temperature of the hydrocarbon feedstream and the hydrogen gas feedstream using a first heat exchanger; (b) combining the feedstreams and expanding the combined feedstream in an expander to decrease the pressure and/or temperature of the combined feedstream; (c) increasing the temperature of the combined feedstream via a second heat exchanger; (d) feeding the combined feedstream into a reactor to produce a reactor effluent; (e) decreasing the temperature of the reactor effluent; and (f) compressing the reactor effluent in a compressor, wherein the expansion of the combined feedstream drives the compressor. Embodiment 2 is the method of embodiment 1, wherein the hydrocarbon feedstream comprises ethane and/or the reactor effluent comprises ethylene. Embodiment 3 is the method of embodiment 1, wherein the hydrocarbon feedstream comprises propane and/or the reactor effluent comprises propylene. Embodiment 4 is the method of any of embodiments 1 to 3, wherein the reactor effluent is expanded in a reactor effluent expander prior to reduction in the temperature of the reactor effluent. Embodiment 5 is the method of any of embodiments of any of 1 to 4, wherein the gas feedstream further comprises a gas selected from the group consisting of steam, nitrogen, methane, hydrogen, carbon dioxide and a combination thereof. Embodiment 6 is the method of embodiments 1 to 5, wherein the gas feedstream and/or the hydrocarbon feedstream has a pressure of about 10 to 50 bar absolute and/or a temperature of about 10° C. to about 100° C. prior to increasing the temperature of the hydrocarbon feedstream and the hydrogen gas feedstream via a first heat exchanger. Embodiment 7 is a method for producing ethylene using a feedstream including ethane and hydrogen including the steps of (a) increasing the temperature of the feedstream via a first heat exchanger to a temperature equal to or greater than about 500° C.; (b) expanding the feedstream in an expander to a pressure of about 4 bar absolute; (c) increasing the temperature of the feedstream to about 500° C. to about 700° C. via a second heat exchange; (d) feeding the feedstream into a reactor and cracking the feedstream at a temperature from about 700° C. to about 880° C. to produce a reactor effluent comprising ethylene at a temperature from about 700° C. to about 880° C. and/or a pressure from about 1 to about 5 bar absolute; (e) decreasing the temperature of the reactor effluent; and (f) compressing the reactor effluent in a compressor to a pressure from about 0.3 bar to about 35 bar absolute, wherein the expansion of the feedstream drives the compressor. Embodiment 8 is the method of embodiment 7 further comprising separating ethylene from the reactor effluent to produce an ethylene product stream. Embodiment 9 is the method of any of embodiments 7 or 8, wherein the temperature of the reactor effluent is about 20° C. to 50° C. prior to compressing the reactor effluent. Embodiment 10 is a method for producing ethylene using a feedstream including ethane and hydrogen, the method including (a) increasing the temperature of the feedstream via a first heat exchanger; (b) expanding the feedstream in a reactor feed expander; (c) increasing the temperature of the feedstream in a second heat exchanger; (d) feeding the feedstream into a reactor and cracking the feedstream at a temperature from about 700° C. to about 880° C. to produce a reactor effluent comprising ethylene; (e) decreasing the temperature of the reactor effluent; and (f) compressing the reactor effluent in a compressor, wherein the expansion of the feedstream drives the compressor. Embodiment 11 is a system for producing olefins using a hydrocarbon feedstream and a gas feedstream including hydrogen, the system including: (a) a first heat exchanger for increasing the temperature of the hydrocarbon feedstream and the hydrogen gas feedstream; (b) a reactants expander, coupled to the first heat exchanger, for decreasing the pressure and/or temperature of the feedstreams; (c) a second heat exchanger, coupled to the reactants expander, for increasing the temperature of the feedstreams via a second heat exchanger; (d) a reactor, coupled to the second heat exchanger and the first heat exchanger, for producing a reactor effluent from the feedstream; (e) a third heat exchanger, coupled to the first heat exchanger, for decreasing the temperature of the reactor effluent; and (f) a compressor, coupled to the third heat exchanger and the reactants expander, for compressing the reactor effluent in a compressor, wherein the expansion of the feedstreams drives the compressor. Embodiment 12 is a system for producing olefins using a hydrocarbon feedstream and a gas feedstream including hydrogen, the system including: (a) a first heat exchanger for increasing the temperature of the hydrocarbon feedstream and the hydrogen gas feedstream; (b) a reactants expander, coupled to the first heat exchanger, for decreasing the pressure and/or temperature of the feedstreams; (c) a second heat exchanger, coupled to the expander, for increasing the temperature of the feedstreams via a second heat exchanger; (d) a reactor, coupled to the second heat exchanger, for producing a reactor effluent from the feedstream; (e) a reactor effluent expander, coupled to the reactor and the first heat exchanger, for decreasing the temperature and/or pressure of the reactor effluent; (f) a third heat exchanger, coupled to the first heat exchanger, for decreasing the temperature of the reactor effluent; and (g) a compressor, coupled to the third heat exchanger, the reactor effluent expander and the reactants expander, for compressing the reactor effluent in a compressor, wherein the expansion of the feedstreams and/or reactor effluent drives the compressor.

### BRIEF DESCRIPTION OF THE DRAWINGS

FIG. 1 is a schematic diagram depicting an exemplary system in accordance with one non-limiting embodiment of the disclosed subject matter.

FIG. 2 is a schematic diagram depicting an exemplary system in accordance with one non-limiting embodiment of the disclosed subject matter.

FIG. 3 is a schematic diagram depicting an exemplary system in accordance with one non-limiting embodiment of the disclosed subject matter.

FIG. 4 is a schematic diagram depicting an exemplary method in accordance with one non-limiting embodiment of the disclosed subject matter.

FIG. 5 depicts a Q-T diagram for an exemplary method in accordance with one non-limiting embodiment of the disclosed subject matter in comparison to a steam cracking method known in the art.

### DETAILED DESCRIPTION

The presently disclosed subject matter provides methods and systems for olefin production from a hydrocarbon feedstream, e.g., by hydropyrolysis, and relates to techniques for improving energy conversion from the heat available in the hydrocarbon feedstream. In certain embodiments, the methods and systems of the present disclosure use a reactants expander for recovering energy from the reactants as mechanical work, e.g., which can be used to drive other components within the system, and to minimize the energy expended during the production of olefin-containing hydrocarbon streams.

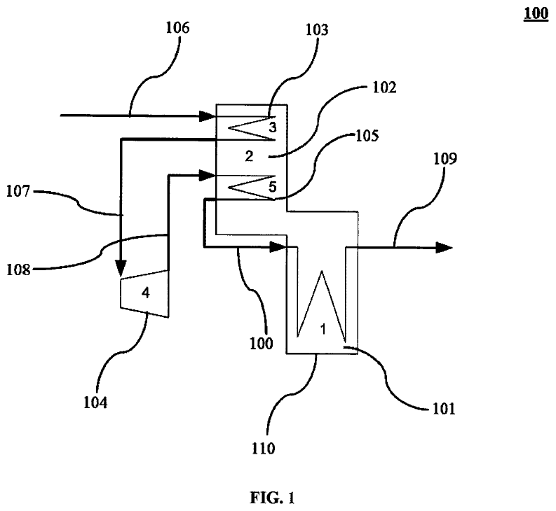

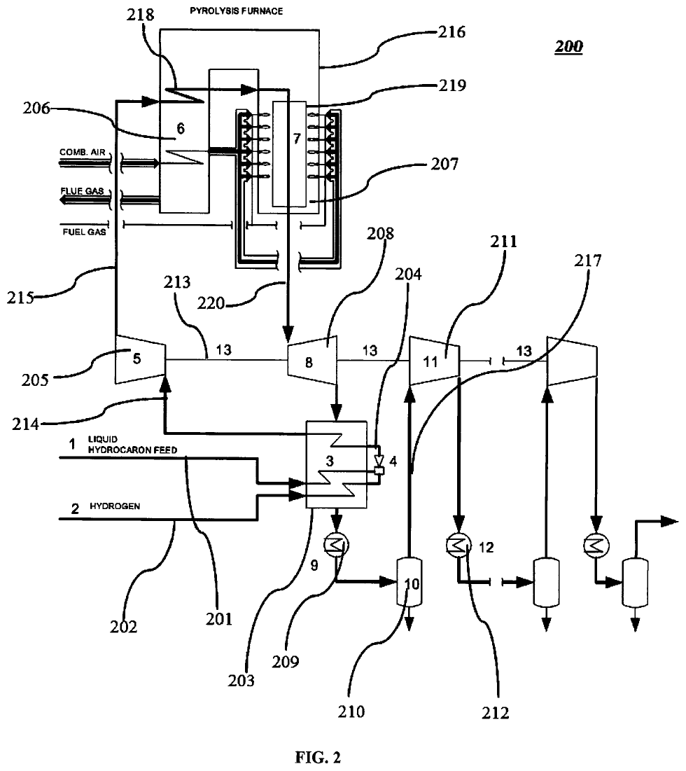

For the purpose of illustration and not limitation, FIGS. 1 and 2 are schematic representations of systems according to non-limiting embodiments of the disclosed subject matter. In certain embodiments and as shown in FIGS. 1 and 2, the system 100 or 200 can include a process furnace 110 or 216. The furnace for use in the present subject matter 110 or 216 can include a radiant section 101 or 207 and a convection section 102 or 206, and can be any furnace known in the art.

As shown in FIG. 1, the system 100 can further include two or more heat exchangers 103 and 105 within the furnace 110, e.g., within the convection section 102 of the furnace 110. In certain embodiments, the heat exchangers can be used to exchange heat between the feedstream containing the reactants, e.g., hydrocarbons, and the flue gases originating from the radiant section 101 of the furnace 110. For example, and not by way of limitation, a feedstream can be provided to a first heat exchanger, e.g., 103, by a feed line 106 that is coupled to the heat exchanger. The heat exchangers of the present disclosure can be of various designs known in the art. In certain embodiments, the heat exchangers can be double pipe exchangers. In certain embodiments, the heat exchangers can include a bundle of tubes housed in a shell, such that streams to be warmed or cooled within the heat exchanger flow through the shell and/or bundle of tubes. In certain embodiments, the heat exchangers can include corrosion-resistant materials. In certain embodiments, the heat exchangers can include an alloy, e.g., steel or carbon steel. In certain embodiments, the heat exchangers can include brazed aluminum.

“Coupled” as used herein refers to the connection of a system component to another system component by any means known in the art. The type of coupling used to connect two or more system components can depend on the scale and operability of the system. For example, and not by way of limitation, coupling of two or more components of a system can include one or more joints, valves, transfer lines or sealing elements. Non-limiting examples of joints include threaded joints, soldered joints, welded joints, compression joints and mechanical joints. Non-limiting examples of fittings include coupling fittings, reducing coupling fittings, union fittings, tee fittings, cross fittings and flange fittings. Non-limiting examples of valves include gate valves, globe valves, ball valves, butterfly valves and check valves.

In certain embodiments, one or more of the heat exchangers, e.g., 103, can be coupled to an expansion element 104, e.g., an expander or an expansion turbine. For example, and not by way of limitation, the heat exchanger 103 can be coupled to the expander 104 via a transfer line 107, e.g., to transfer the feedstream from the heat exchanger 103 to the expander 104 (also referred to herein as a reactants expander). In certain embodiments, the feedstream can be expanded within the expander 104 to reduce its pressure. Additionally, the expander can result in the feedstream having an increased volume and decreased temperature, as described below. The expander can be used to extract energy from the feedstream in the form of mechanical work, which can be used to drive other components within the system. For example, and not by way of limitation, the reactants expander 104 can be coupled to a compressor, which can be used to compress the effluent that exits the reactor (also referred to herein as the reactor effluent). In certain embodiments, the reactor can be coupled to the furnace 110. In certain embodiments, the reactor expander can be coupled to a generator, e.g., to generate electricity from the energy extracted from the reactor expander. Alternatively or additionally, the reactor expander can be coupled to a refrigeration compressor. Non-limiting examples of refrigeration compressors include ethylene refrigerant compressors, propylene refrigerant compressors, propane refrigerant compressors and mixed refrigerant compressors (e.g., refrigerants can be a C1/C2 mix, a C2/C3 mix, a C1/C3 mix or a C1/C2/C3 mix).

In certain embodiments, the reactants expander 104 can be coupled to a second heat exchanger 105 within the convection section 102 of the furnace 110. For example, and not by way of limitation, the expander 104 can be coupled to the second heat exchanger 105 via transfer line 108. In certain embodiments, the expanded feedstream can be transferred to the second heat exchanger 105 via transfer line 108 from the expander 104. In certain embodiments, the temperature of the expanded feedstream can be increased by exchanging heat with flue gases originating from the radiant section 101 of the furnace 110 via the second heat exchanger 105. In certain embodiments, the second heat exchanger 105 can be coupled to the reactor, e.g., for transferring the heated feedstream to the reactor.

For the purpose of illustration and not limitation, FIG. 2 is a schematic representation of a system according to a non-limiting embodiment of the disclosed subject matter. In certain embodiments, the system 200 can include one or more feed lines, e.g., 201 and 202, coupled to a heat exchanger 203. For example, and not by way of limitation, feed line 201 can be used to feed a hydrocarbon feedstream to the heat exchanger 203. Alternatively or additionally, feed line 202 can be used to transfer a second feedstream and/or diluent, e.g., a hydrogen stream, to the heat exchanger 203. In certain embodiments, the system 200 can further include a chamber 204, e.g., mixing chamber, coupled to the heat exchanger 203. The chamber 204 can be used for combining the multiple feedstreams fed into the heat exchanger 203, e.g., by feed lines 201 and 202, into a single feedstream after the initial heat exchange.

In certain embodiments, the system 200 can further include an expansion element 205, e.g., an expander or an expansion turbine. For example, and not by way of limitation, the heat exchanger 203 can be coupled to the expander 205 via a transfer line 214, e.g., to transfer the feedstream, e.g., the combined feedstream as disclosed above, from the heat exchanger 203 to the expander 205. In certain embodiments, the expander 205 can be used to reduce the pressure and/or temperature of the feedstream and for extracting work from the heat of the feedstream as described above.

The system 200 can further include a second heat exchanger 218 coupled to the expander 205. The second heat exchanger 218 can be located within the convection section 206 of the furnace 216 and can be coupled to the expander 205 via transfer line 215. In certain embodiments, the expanded feedstream can be transferred from the expander 205 and to the heat exchanger 218 via transfer line 215 to increase the temperature of the expanded feedstream by exchanging heat with one or more gases within the furnace. For example, the expanded feedstream can exchange heat with one or more flue gases originating from the radiant section 207 of the furnace 216.

In certain embodiments, the system 200 can further include a reactor 219 coupled to the second heat exchanger 218. In certain embodiments, the reactor 219 can be any reactor that can be operated at low pressures, e.g., at pressures below about 45 bar gauge (bar_{g}), and/or at high temperatures, e.g., at temperatures higher than about 500° C. In certain embodiments, the reactor 219 can be a reactor that is used to produce olefins, e.g., by cracking hydrocarbon containing feedstreams. In certain embodiments, the reactor 219 can be a reactor used to produce propylene, butylene, 1,3-butadiene and/or ethylene. In certain embodiments, the reactor 219 can be a reactor used for steam reforming for the production of hydrogen, e.g., during syngas production. In certain embodiments, the reactor 219 can be a reactor used for exothermic processes, e.g., hydrocracking such as, but not limited to, mild hydrocracking. For example, and not by way of limitation, the reactor 219 can be a reactor used for mild hydrocracking of pyrolysis gas to generate benzene, toluene, xylene and/or ethylbenzene.

In certain embodiments, the reactor 219 can be coupled to a second expansion element 208. For example, and not by way of limitation, the reactor 219 can be coupled to the second expander 208 via a transfer line 220, e.g., to transfer the effluent from the reactor to the expander 208 (also referred to herein as a reactor effluent expander). In certain embodiments, the reactor effluent can be expanded within the reactor effluent expander 208 to reduce its pressure and/or decrease its temperature. The reactor effluent expander can be used to extract work from the heat of the reactor effluent to drive other components within the system. Alternatively, the reactor 219 can be directly coupled to a heat exchanger, e.g., 203, to reduce the temperature of the reactor effluent without the use of a reactor effluent expander.

In certain embodiments, the reactor effluent expander 208 can be coupled to a heat exchanger to further reduce the temperature of the reactor effluent. For example, and not by way of limitation, the reactor effluent expander 208 can be coupled to the first heat exchanger 203 to exchange heat between the reactor effluent and the feedstreams within the first heat exchanger 203. In certain embodiments, the system can include a third heat exchanger, e.g., for further reducing the temperature of the reactor effluent. In certain embodiments, the first heat exchanger 203 can be coupled to the third heat exchanger 209.

In certain embodiments where condensation of components within the reactor effluent occurs following cooling, the system 200 can further include one or more separation units 210 for separating the condensed components from the reactor effluent. The separation unit 210 can be coupled to the third heat exchanger 209.

In certain embodiments, the system 200 can further include one or more compressors 211. The compressor 211 can be coupled to the separation unit 210 via a transfer line 217 and/or coupled to the third heat exchanger 209 to increase the pressure of the reactor effluent, e.g., to prepare the reactor effluent for downstream separation processes. The compressor 211 can include one or more stages and/or one or more cooling units 212 (see FIG. 2).

In certain embodiments, the reactants expander 205, reactor effluent expander 208 and/or the one or more stages of the compressor 211 can be mounted on the same axis 213 to allow for the transfer of mechanical work between these components. For example, and not by way of limitation, the compressor 211 can be coupled to the reactants expander 205 and/or the reactor effluent expander 208.

The system of the present disclosure can further include additional components and accessories including, but not limited to, one or more additional feed lines, gas exhaust lines, cyclones, product discharge lines, reaction zones, heating elements and one or more measurement accessories. The one or more measurement accessories can be any suitable measurement accessory known to one of ordinary skill in the art including, but not limited to, pH meters, pressure indicators, pressure transmitters, thermowells, temperature-indicating controllers, gas detectors, analyzers and viscometers. The components and accessories can be placed at various locations within the system.

In accordance with the embodiments of the subject matter previously described, the system and the various components and accessories that can be included in the system, e.g., reactants expander, can be made out of a plurality of suitable materials. Suitable materials include, but are not limited to, aluminum, stainless steel, carbon steel, glass-lined materials, polymer-based materials, nickel-based metal alloys, titanium-based alloys, cobalt-based metal alloys or combinations thereof. Additional non-limiting example of suitable materials include chromium, hafnium, niobium, platinum, rare earth metals, rhenium, ruthenium, tantalum, titanium, tungsten and vanadium. In certain embodiments, such metals can be present within a metal alloy, e.g., a nickel-based alloy.

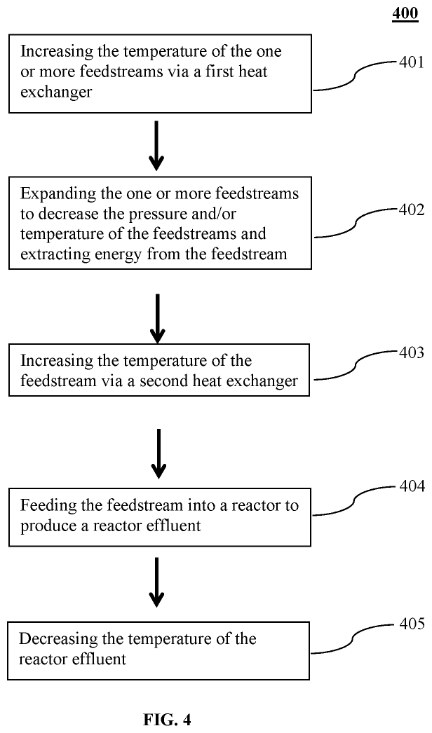

The presently disclosed subject matter provides methods for the production of olefins from a hydrocarbon feedstream and the extraction of heat energy from the hydrocarbon feedstream to drive downstream steps of the methods. For the purpose of illustration and not limitation, FIG. 4 is a schematic representation of a method according to a non-limiting embodiment of the disclosed subject matter. In certain embodiments, the method can include providing one or more feedstreams. For example, the method 400 can include providing at least two feedstreams, e.g., a first feedstream and a second feedstream. In certain embodiments, the first feedstream can be a hydrocarbon stream, e.g., a liquid hydrocarbon stream. Non-limiting examples of such hydrocarbon streams include naphtha streams, C4 hydrocarbon streams, ethane-containing streams, 1,3-butadiene-containing streams and propane-containing streams. In certain embodiments, the second feedstream can be a gaseous feedstream, e.g., that includes hydrogen, steam, nitrogen or a combination thereof. In certain embodiments, the one or more feedstreams can include methane, gas oil, vacuum gas oil, vacuum residue, synthesis gas, Fischer-Tropsch liquids/waxes and/or pyrolysis gasoline. In certain embodiments, the pyrolysis gasoline can be obtained from a steam cracking process.

The method 400 can include increasing the temperature of the one or more feedstreams 401. In certain embodiments, the temperature of the one or more feedstreams can be increased by exchanging heat between the feedstreams and an additional stream to increase the temperature of the feedstreams 401. Heat exchange between the feedstreams and the additional stream can occur with a first heat exchanger. For example, the temperature of the feedstreams can be increased by the exchanging of heat with a stream such as, but not limited to, a flue gas or an effluent stream exiting a chemical reactor. In certain embodiments, prior to exchanging heat with the additional stream, the feedstreams can have a temperature from about 10° C. to about 150° C. In certain embodiments, the temperature of the feedstreams prior to heat exchange can be about 80° C. In certain embodiments, after exchanging heat with the additional stream, the temperature of the feedstreams can be of a temperature close to the boiling point of one or more components within the feedstream and/or of a temperature that results in the partial evaporation of the feedstream. In certain embodiments, the temperature of the feedstreams can be from about 300° C. to about 600° C. after heat exchange.

In certain embodiments where two or more feedstreams are subjected to heat exchange, the method 400 can further include combining the two or more feedstreams into a single feedstream and/or subjecting the single feedstream to a second heat exchange with the additional stream within the first heat exchanger (see FIG. 2). In certain embodiments, after exchanging heat with the additional stream for a second time, the temperature of the combined feedstream can be of a temperature that results in the evaporation of the feedstream, e.g., the heated combined feedstream can have a vapor fraction greater than about 90%, greater than about 91%, greater than about 92%, greater than about 93%, greater than about 94%, greater than about 95% or greater than about 96%. In certain embodiments, the one or more feedstreams can have a pressure of about 10 to about 50 bar absolute (barₐ) prior to and/or after heat exchange within the first heat exchanger. For example, and not by way of limitation, the one or more feedstreams can have a pressure of about 30 barₐ.

As used herein, the term “about” or “approximately” means within an acceptable error range for the particular value as determined by one of ordinary skill in the art, which will depend in part on how the value is measured or determined, i.e., the limitations of the measurement system. For example, “about” can mean a range of up to 20%, up to 10%, up to 5% and/or up to 1% of a given value.

In certain embodiments, the method 400 can further include expanding the feedstream, e.g., the combined feedstream, to decrease the pressure and/or temperature of the feedstream 402. The feedstream can be expanded within an expander, e.g., a expander turbine as disclosed above. Following expansion, the feedstream can have a pressure from about 1 barₐ to about 20 barₐ, e.g., a pressure of about 4 barₐ, and can have a vapor fraction greater than about 90%, greater than about 91%, greater than about 92%, greater than about 93%, greater than about 94%, greater than about 95% or greater than about 96%. In certain embodiments, the temperature of the feedstream can be from about 300° C. to about 500° C. following expansion. In the disclosed subject matter, the energy obtained during expansion of the feedstreams in the expander can be converted into mechanical energy that can be used to drive other system components used within the disclosed methods. For example, and not by way of limitation, the extracted energy can be used to drive a compressor, e.g., in common drive with the reactants expander and/or the reactor effluent expander, which can be used to compress the reactor effluent that are produced within a chemical reactor used in the disclosed method. Non-limiting examples of methods for transferring the work obtained from the expansion of the feedstream and/or reactor effluent, described below, can include the use of gears, an electric generator, an electric motor, a hydraulic pump and/or motor or a pneumatic pump and/or motor. In certain embodiments, the work can be transferred by coupling the system component to the same axis as the reactants expander (e.g., mechanical coupling).

The method can further include increasing the temperature of the feedstream 403. In certain embodiments, the temperature of the feedstream can be increased by heat exchange within a second heat exchanger. For example, and not by way of limitation, the heat exchanger can be located within a process furnace, e.g., within the convection section of the process furnace, and/or the feedstream can exchange heat with one or more gas streams within the furnace. In certain embodiments, the feedstream can exchange heat with one or more flue gases produced within the reactor, e.g., within the radiation section of the furnace (see FIG. 2). In certain embodiments, the feedstream can exchange heat with the reactor effluent and/or the feedstream can exchange heat with the exhaust gases (or the waste heat recovered therefore) of a turbine, e.g., a gas turbine. Following heat exchange within the second heat exchanger, the feedstream can have a temperature from about 500° C. to about 700° C.

In certain embodiments, the method can further include feeding the feedstream into a reactor to produce a product stream (also referred to herein as the reactor effluent) that includes the hydrocarbon reaction products 404. For example, and not by way of limitation, the methods of the disclosed subject matter can be used to produce a reactor effluent that includes ethylene by using a hydrocarbon feedstream that includes ethane and cracking the ethane within the reactor. In certain embodiments, the hydrocarbon feedstream can include butane and/or the reactor effluent can include 1-butene, isobutylene, hydrogen or a combination thereof. In a non-limiting embodiment, the hydrocarbon feedstream can be a C4 hydrocarbon stream and/or the reactor effluent can include 1,3-butadiene. In certain embodiments, the hydrocarbon feedstream can be a kerosene fraction and/or the reactor effluent can include 1-butene, ethylene, methane, acetylene, ethane, propylene, propane, 1,3-butadiene, 1-butene, hydrogen or a combination thereof. In certain embodiments, the hydrocarbon feedstream can include propane and/or the reactor effluent can include hydrogen, methane, ethane, ethylene, propane and/or propylene or a combination thereof. In certain embodiments, the feedstream can include methane and steam and/or the reactor effluent can include hydrogen, carbon monoxide, carbon dioxide and/or steam. In certain embodiments, the feedstream can include pyrolysis gas and/or hydrogen and/or the reactor effluent can include hydrogen, methane, ethane, propane, butane, pentane, benzene, toluene, xylene and/or ethyl benzene. In certain embodiments, the feedstream can include naphtha, gas oil, vacuum gas oil and/or vacuum residue and/or the reactor effluent can include hydrogen, methane, ethane, ethylene, propylene, 1-butene, 2-butene, 1,3-butadiene, pyrolysis gas (pygas) and/or C9+ hydrocarbons.

In certain embodiments, the reactor and/or the combustion air can be heated using the radiant heat produced by the process furnace, e.g., produced within the radiation section of the furnace. For example, the reactor or components of the reactor can be positioned within the radiant section of the furnace. In certain embodiments, the reactor effluent exiting the reactor can have a temperature of about 750° C. to about 880° C. and/or a pressure of about 2 barₐ to about 5 barₐ. In certain embodiments, the reactor effluent can have a temperature of about 850° C. and/or a pressure of about 3 barₐ. In certain embodiments, the reactor effluent can have a temperature of about 850° C. and/or a pressure in the range of about 2 barₐ to about 45 barₐ.

In certain embodiments, the method can include decreasing the temperature of the reactor effluent 405. In certain embodiments, the temperature of the reactor effluent can be decreased by exchanging heat within one or more heat exchangers. Following heat exchange, the reactor effluent can have a final temperature of about 20° C. to about 50° C. and/or a final pressure of about 0.1 barₐ to about 2 barₐ. In certain embodiments, the reactor effluent can undergo multiple heat exchanges to have a final temperature and/or pressure as described above. For example, and not by way of limitation, the reactor effluent can exchange heat with the one or more feedstreams within the first heat exchanger. Following heat exchange within the first heat exchanger, the reactor effluent can have a temperature from about 300° C. to about 100° C. and/or a pressure of about 3 barₐ to about 0.1 barₐ, e.g., a temperature of about 130° C. and/or a pressure of about 0.3. In certain embodiments, the reactor effluent exiting the first heat exchanger can be further cooled within a third heat exchanger (see FIG. 2), e.g., by direct or indirect water cooling. In certain embodiments, the reactor effluent can have a temperature of about 20° C. to about 30° C. and/or a final pressure of about 0.1 barₐ to about 1 barₐ upon exiting the third heat exchanger, e.g., a temperature of about 30° C. and/or a pressure of about 0.3 barₐ.

Alternatively or additionally, prior to decreasing the temperature of the reactor effluent within the one or more heat exchangers, e.g., within the first heat exchanger and/or the third heat exchanger, the reactor effluent can be expanded in a reactor effluent expander to decrease the temperature and/or pressure of the reactor effluent (see FIG. 2). In certain embodiments, upon expansion, the temperature of the reactor effluent can be decreased to about 600° C. to about 700° C., e.g., 630° C., and/or have a pressure from about 1.2 barₐ to 0.2 barₐ, e.g., 0.3 barₐ. The temperature and/or pressure of the reactor effluent exiting the reactor effluent expander can depend on the on the amount of cooling required, the inlet pressure of the reactor effluent and/or the inlet temperature of the reactor effluent. In certain embodiments, a method of the present disclosure does not include expanding the reactor effluent within a reactor effluent expander.

In certain embodiments where the reactor effluent is expanded, the reactor effluent can be subjected to heat exchange prior to expansion within the reactor effluent expander. For example, and not by way of limitation, the temperature of the reactor effluent can be reduced upstream from the reactor effluent expander. In certain embodiments, the temperature of the reactor effluent can be reduced by means of direct quenching with a gas or liquid or indirect quenching through heat exchange, e.g., by steam generation in a primary transfer line exchanger. Cooling the reactor effluent prior to transferring the reactor effluent to the reactor effluent expander can allow the expander to operate at a lower inlet temperature and under less severe operating conditions which can allow the reactor effluent expander to be constructed of different types of materials.

In certain embodiments, following the reduction in the temperature of the reactor effluent, the method can include separating the components within the reactor effluent that condensed, if any, upon cooling.

In certain embodiments, the method can further include compressing the reactor effluent to increase the pressure of the effluent 406. In certain embodiments, the pressure of the reactor effluent following compression can be from about 0.3 barₐ to about 40 barₐ to allow downstream separation of the hydrocarbons products from the reactor effluent. The type of separation processes used in the disclosed method depends on the types of hydrocarbons that are present within the reactor effluent. Non-limiting examples of separation processes that can be used in the disclosed methods are disclosed in U.S. Pat. Nos. 5,979,177, 6,637,237 and 6,705,113, EP2326899 and WO 2010/016815. One non-limiting embodiment of a separation process that can be used in the disclosed methods for the production of ethylene includes washing the reactor effluent after compression, drying and cooling the reactor effluent in a cold box, e.g., cooling can be provided from cold recovery of the products after separation and the use of an ethylene and propylene refrigerant system, to a temperature in the range of about −150 to about −180° C. The condensed reactor effluent stream can be separated into C1, C2, C3, C4 and C5+ fractions in a series of distillation columns that include a demethanizer, a deethanizer, a depropanizer and a debutanizer. The deethanizer can produce a C2 fraction containing acetylene, ethylene and ethane. In certain embodiments, the acetylene can be extracted or hydrogenated to ethylene and the ethylene and ethane can be separated from the C2 fraction in a C2 splitter distillation column.

The following examples are merely illustrative of the presently disclosed subject matter and should not be considered as limitations in any way.

### EXAMPLES

### Example 1: Conversion of Heat Energy to Mechanical Energy by Use of an Expander

This example depicts the use of an reactants expander to convert heat energy from a feedstream into mechanical work. A simulation using the software Aspen Plus version 8.2 (Aspen Technology, Inc.) was performed to model the process. As shown in FIG. 1, a steam cracking furnace includes a radiant section (1), where the main heat transfer to the reactor occurs through radiation and a convection section (2), where the heat available in the hot flue gasses leaving the radiant section is transferred. The reactor feed and water/steam mixture was at a pressure of 13 barₐ and preheated to 450° C. through heat transfer in a heat exchanger (3) in the convection section (2) of the process furnace and expanded in a turbine (4) to 5 barₐ. The use of the turbine (4) yielded 595 kW_{mech} of work at the cost of 595 kWₜₕ heat used to heat the reactor feed. As a result of the expander, the reactor feed mixture now has a lower temperature, a lower pressure and increased volume. The work generated by the turbine (4) can be used to drive any rotating equipment in the steam cracker such as a compressor, or pump directly (by mechanically coupling the equipment), or indirectly by generating electricity from the turbine and converting this electricity to work with an electric motor. The feed mixture leaving the turbine (4) was then be reheated a heat exchanger (5) to 600° C. with heat from the hot flue gasses originating from the radiant section (1).

In certain steam cracking methods known in the art, additional heat needs to be supplied to the feed/water/steam mixture. This heat can originate from the flue gasses leaving the radiation section of the furnace, the reactor effluent or from the steam generated. Due to the additional heating of the hydrocarbon, the steam production of the furnace will be lower. In this case, 595 kWₜₕ less steam was generated while delivering 595 kW_{mech} work, and from the energy contained by the steam, only approximately 30% will become work.

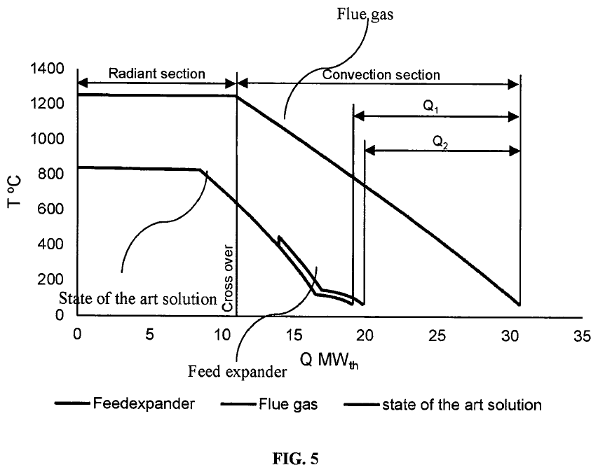

Using the system shown in FIG. 1, where the process furnace is equipped with an expander turbine and reheat exchanger, the heat energy obtained from the feedstream can be converted into mechanical work with high efficiency. As shown in the QT-diagram of FIG. 5, the orange line depicts the heat input to the feed/water mixture in case a feed expander is applied. The consequence of this is that less heat is available for heat recovery (Q₂<Q₁). However, this difference (Q₁−Q₂) is converted to mechanical work with high efficiency (typically >80%) as compared to methods known in the art.

### Example 2: Hydropyrolysis of a Kerosene Fraction to Produce Ethylene

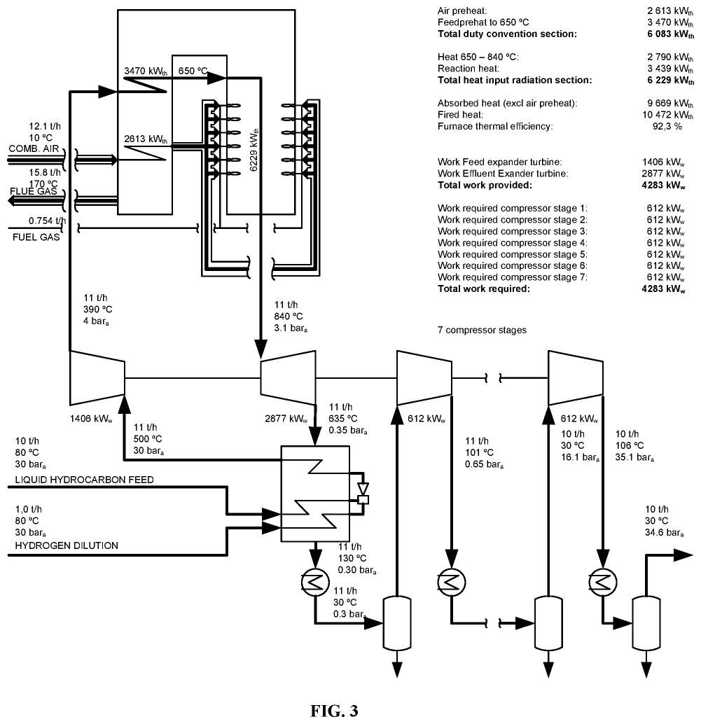

This example provides details regarding a hydropyrolysis method for cracking a kerosene fraction using the disclosed subject matter as compared to a steam cracking process known in the art. A simulation using the software Aspen Plus version 8.2 (Aspen Technology, Inc.) was performed to model the method. As shown in FIGS. 2 and 3, a liquid hydrocarbon feed (1) with a pressure of 30 bar absolute (barₐ) and an original temperature of 80° C. was heated close to the boiling point or partly evaporated with heat originating from the reactor effluent by means of a heat exchanger (3). The liquid hydrocarbon stream was straight run kerosene that has a boiling point range from 150° C. to 300° C. Following an initial heat exchange within the heat exchanger (3), the hydrocarbon feedstream had a temperature of 300° C. A stream of hydrogen (2) that had a pressure of 30 barₐ and an original temperature of 80° C. was heated with heat originating from the reactor effluent by means of a heat exchanger (3) to such a temperature that after mixing with the liquid or partly evaporated hydrocarbon feed in a mixing device (4), the mixture was fully evaporated. Following an initial heat exchange within the heat exchanger (3), the hydrogen stream had a temperature of 300° C. The hydrogen-hydrocarbon mixture was further heated in a heat exchanger system (3) to a temperature of 500° C. so that after expansion in a reactor feed expander (5), the outlet stream was 95% vapor or more.

The heated hydrogen-hydrocarbon mixture was expanded in the reactor feed expander (5) to a reduced pressure of 4 barₐ and a temperature of 390° C. The effluent from the reactor feed expander was reheated in the convection section of the process furnace by drawing heat from the hot flue gasses leaving the radiation section (7) of the furnace. After reheating the reactor feed to a temperature of 650° C., the hydrogen-hydrocarbon mixture was cracked in a tubular reactor heated by radiant heat in the radiant section (7) of a process furnace. Heat to the reactor was supplied by a furnace with mainly radiant heat transfer to the tubular reactor in the radiation section (7), and the required combustion air was preheated by the energy present in the flue gasses in the convection section (6) of the furnace. The reactor effluent was then transferred from the tubular reactor at a temperature of 840° C. and a pressure of 3.1 barₐ.

The reactor effluent from the radiant section (7) of the process furnace was expanded in a reactor effluent expander (8) to a pressure of 0.35 barₐ. The pressure can also be higher if less cooling is required or if the inlet pressure was higher. It can also be lower if more cooling is required or more work needs to be generated. The remainder of the heat present in the reactor effluent that exited the reactor effluent expander (8) was recovered by the heat exchanger (3) to a temperature 130° C. and cooled to a temperature 30° C. by means of direct or indirect water cooling (9). Condensed components were separated out (10) and the gas was compressed in a compressor (11) to a higher pressure to allow for downstream separation. The compressor (11) included several stages with interstage coolers (12). The reactor feed expander (5), reactor effluent expander (8) and all or some stages of the compressor (11) were mounted on the same axis to allow for the transfer of mechanical work between these devices.

Methods for steam cracking of a kerosene fraction known in the art include cracking 10 t/h of a kerosene fraction at 818° C. COT and 1.7 barₐ COP, with a dilution of 3.5 t/h of steam. In the disclosed hydropyrolysis method as shown in FIG. 3, 10 t/h of a kerosene fraction was cracked at 818° C. COT and 2.8 barₐ COP mixed with 1.0 t/h of hydrogen feed.

The presently disclosed method consumed less energy compared to methods known in the art. For example, in the disclosed method, 0.754 t/h of fuel was used (which is equivalent to 38 GJ/h) as compared to 41 GJ/h that was used in a method known in the art. Further, the energy requirements for the furnace plus charge gas compressor section was approximately 41/4.54=9.0 GJ per tonne ethylene plus propylene for the steam cracking process (method known in the art), whereas it is 38/4.91=7.7 GJ per tonne ethylene plus propylene for the disclosed hydropyrolysis process. Therefore, the methods of the disclosed subject matter are more energy efficient than methods known in the art.

In addition, use of hydrogen as the diluent instead of steam resulted in the production of light hydrocarbon components from the feed (Table 1). Although ethane was produced in both the steam cracking process as well as the hydropyrolysis process, it was assumed to be recycled in an ethane steam cracking furnace under suitable conditions for ethane steam cracking with 75 wt % ultimate yield of ethylene from ethane and is indicated as 0 wt % in Table 1. In addition, considering the disclosed hydropyrolysis method consumes hydrogen, the weight percent for hydrogen is indicated as negative (Table 1). As shown in Table 1, the yield of ethylene and propylene from a liquid feedstock using the disclosed method was higher compared to steam cracking. In addition, the use of hydrogen in the disclosed hydropyrolysis process as the diluent can suppress coke formation in the reactor tube.

TABLE 1

| Method | Steam cracking method | Hydropyrolysis method |
|---|---|---|
| ultimate yield | % wt | % wt |
| Hydrogen | 2.2 | −1.2 |
| methane | 14.9 | 23.0 |
| ethylene | 31.3 | 40.5 |
| Ethane | 0 | 0 |
| propylene | 14.1 | 8.7 |
| C2= + C3= total | 45.4 | 49.1 |

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed. Additionally, it will be apparent to those skilled in the art that various modifications and variations can be made in the compositions and methods of the disclosed subject matter without departing from the spirit or scope of the disclosed subject matter. Thus, it is intended that the disclosed subject matter include modifications and variations that are within the scope of the appended claims and their equivalents.

Various patents and patent applications are cited herein, the contents of which are hereby incorporated by reference herein in their entireties.

1. A method for producing olefins using a hydrocarbon feedstream and a gas feedstream including hydrogen, the method comprising:
(a) increasing the temperature of the hydrocarbon feedstream and the hydrogen gas feedstream using a first heat exchanger;
(b) combining the feedstreams and expanding the combined feedstream in an expander to decrease the pressure and/or temperature of the combined feedstream;
(c) increasing the temperature of the combined feedstream via a second heat exchanger;
(d) feeding the combined feedstream into a reactor to produce a reactor effluent;
(e) decreasing the temperature of the reactor effluent; and
(f) compressing the reactor effluent in a compressor,
wherein the expansion of the combined feedstream drives the compressor.

2. The method of claim 1, wherein the hydrocarbon feedstream comprises ethane and/or the reactor effluent comprises ethylene.

3. The method of claim 1, wherein the hydrocarbon feedstream comprises propane and/or the reactor effluent comprises propylene.

4. The method of claim 1, wherein the reactor effluent is expanded in a reactor effluent expander prior to reduction in the temperature of the reactor effluent.

5. The method of claim 1, wherein the gas feedstream further comprises a gas selected from the group consisting of steam, nitrogen, methane, hydrogen, carbon dioxide and a combination thereof.

6. The method of claim 1, wherein the gas feedstream and/or the hydrocarbon feedstream has a pressure of about 10 to 50 bar absolute and/or a temperature of about 10° C. to about 100° C. prior to increasing the temperature of the hydrocarbon feedstream and the hydrogen gas feedstream via a first heat exchanger.

7. The method of claim 2, wherein the reactor effluent is expanded in a reactor effluent expander prior to reduction in the temperature of the reactor effluent.

8. The method of claim 1, further comprising separating ethylene from the reactor effluent to produce an ethylene product stream.

9. The method of claim 7, wherein the temperature of the reactor effluent is about 20° C. to 50° C. prior to compressing the reactor effluent.

10. The method of claim 2, wherein the gas feedstream further comprises a gas selected from the group consisting of steam, nitrogen, methane, hydrogen, carbon dioxide and a combination thereof.

11. A system for producing olefins using a hydrocarbon feedstream and a gas feedstream including hydrogen, the method comprising:
(a) a first heat exchanger for increasing the temperature of the hydrocarbon feedstream and the hydrogen gas feedstream;
(b) a reactants expander, coupled to the first heat exchanger, for decreasing the pressure and/or temperature of the feedstreams;
(c) a second heat exchanger, coupled to the reactants expander, for increasing the temperature of the feedstreams via a second heat exchanger;
(d) a reactor, coupled to the second heat exchanger and the first heat exchanger, for producing a reactor effluent from the feedstream;
(e) a third heat exchanger, coupled to the first heat exchanger, for decreasing the temperature of the reactor effluent; and
(f) a compressor, coupled to the third heat exchanger and the reactants expander, for compressing the reactor effluent in a compressor,
wherein the expansion of the feedstreams drives the compressor.

12. A system for producing olefins using a hydrocarbon feedstream and a gas feedstream including hydrogen, the system comprising:
(a) a first heat exchanger for increasing the temperature of the hydrocarbon feedstream and the hydrogen gas feedstream;
(b) a reactants expander, coupled to the first heat exchanger, for decreasing the pressure and/or temperature of the feedstreams;
(c) a second heat exchanger, coupled to the expander, for increasing the temperature of the feedstreams via a second heat exchanger;
(d) a reactor, coupled to the second heat exchanger, for producing a reactor effluent from the feedstream;
(e) a reactor effluent expander, coupled to the reactor and the first heat exchanger, for decreasing the temperature and/or pressure of the reactor effluent;
(f) a third heat exchanger, coupled to the first heat exchanger, for decreasing the temperature of the reactor effluent; and
(g) a compressor, coupled to the third heat exchanger, the reactor effluent expander and the reactants expander, for compressing the reactor effluent in a compressor,
wherein the expansion of the feedstreams and/or reactor effluent drives the compressor.

13. The method of claim 3, wherein the gas feedstream further comprises a gas selected from the group consisting of steam, nitrogen, methane, hydrogen, carbon dioxide and a combination thereof.

14. The method of claim 4, wherein the gas feedstream further comprises a gas selected from the group consisting of steam, nitrogen, methane, hydrogen, carbon dioxide and a combination thereof.

15. The method of claim 3, wherein the gas feedstream and/or the hydrocarbon feedstream has a pressure of about 10 to 50 bar absolute and/or a temperature of about 10° C. to about 100° C. prior to increasing the temperature of the hydrocarbon feedstream and the hydrogen gas feedstream via a first heat exchanger.

16. The method of claim 4, wherein the gas feedstream and/or the hydrocarbon feedstream has a pressure of about 10 to 50 bar absolute and/or a temperature of about 10° C. to about 100° C. prior to increasing the temperature of the hydrocarbon feedstream and the hydrogen gas feedstream via a first heat exchanger.

17. The method of claim 5, wherein the gas feedstream and/or the hydrocarbon feedstream has a pressure of about 10 to 50 bar absolute and/or a temperature of about 10° C. to about 100° C. prior to increasing the temperature of the hydrocarbon feedstream and the hydrogen gas feedstream via a first heat exchanger.

18. The method of claim 2, wherein the gas feedstream and/or the hydrocarbon feedstream has a pressure of about 10 to 50 bar absolute and/or a temperature of about 10° C. to about 100° C. prior to increasing the temperature of the hydrocarbon feedstream and the hydrogen gas feedstream via a first heat exchanger.

19. The method of claim 3, wherein the gas feedstream and/or the hydrocarbon feedstream has a pressure of about 10 to 50 bar absolute and/or a temperature of about 10° C. to about 100° C. prior to increasing the temperature of the hydrocarbon feedstream and the hydrogen gas feedstream via a first heat exchanger.

20. The method of claim 4, wherein the gas feedstream and/or the hydrocarbon feedstream has a pressure of about 10 to 50 bar absolute and/or a temperature of about 10° C. to about 100° C. prior to increasing the temperature of the hydrocarbon feedstream and the hydrogen gas feedstream via a first heat exchanger.
